(19) United States
(12) Patent Application Publication
(10) Pub. No.: US 2025/0381288 A1
(43) Pub. Date: Dec. 18, 2025

(54) Anti-VEGF Single Chain Variable Fusion Protein (RNAT92)

(71) Applicants: Sarfaraz K. Niazi, Deerfield, IL (US); Matthias Magoola, Kampala (UG); Zafeer Ahmad, Wadsworth, IL (US); Zamara Mariam, Coventry City (GB)

(72) Inventors: Sarfaraz K. Niazi, Deerfield, IL (US); Matthias Magoola, Kampala (UG); Zafeer Ahmad, Wadsworth, IL (US); Zamara Mariam, Coventry City (GB)

(21) Appl. No.: 18/744,645

(22) Filed: Jun. 16, 2024

(51) Int. Cl. A61K 47/68

(52) U.S. Cl. CPC A61K 47/6811; A61K 47/6889

(57) ABSTRACT

Anti-VEGF (Vascular Endothelial Growth Factor) (RNAT92) is a single chain variable fusion protein comprising fragment antibody variable light and variable heavy chains with binding domains conjugated with transferrin protein to induce transcytosis across the blood-retina barrier to treat neovascular (wet) age-related macular degeneration (AMD) by systemic administration instead of intravitreal administration, and binding and transcytosis to cancer cells to treat cancer. The present invention can be manufactured by recombinant process or by encoding in vivo as mRNA.

### SEQUENCE LISTING

The instant application contains a Sequence Listing, which has been submitted electronically in ASCII format and is hereby incorporated by reference in its entirety. Said ASCII text file, created on Jun. 10, 2024, is named RNAT92_SequenceListing, and is 3 kb in size.

### BACKGROUND OF THE INVENTION

Anti-VEGF antibodies and fusion proteins are widely used to treat disorders that require injecting them into the eye, a harrowing route with severe multiple side effects and a high cost of treatment; these antibodies can be administered systemically by converting them to single chain variable fragments and conjugating them with transferrin protein. While this invention allows an alternate route to intravitreal administration, it also presents improved anticancer activity by binding to cancer cell surface that has over-expressed transferrin receptors and enters the cancer cells more efficiently.

RNAT92 is a humanized monoclonal single-chain variable fragment (scFv) that binds and inhibits vascular endothelial growth factor A (VEGF-A), decreasing neovascularization in the eye. Thus, it can treat Neovascular Age-related Macular Degeneration (nAMD, wet AMD, or wAMD) and Diabetic Macular Edema (DME). The exact mechanism can help reduce the growth of cancer cells.

Anti-VEGF antibodies and fusion proteins target and inhibit VEGF, a protein crucial for stimulating blood vessel formation (angiogenesis). These antibodies bind to VEGF molecules, preventing them from interacting with their receptors (VEGFR-1 and VEGFR-2) on the surface of endothelial cells, which line the interior of blood vessels. This inhibition blocks the activation of signaling pathways essential for angiogenesis, thus reducing the formation of new blood vessels. This is particularly important in conditions where abnormal blood vessel growth is problematic, such as certain cancers and age-related macular degeneration (AMD). Additionally, by preventing VEGF from increasing vascular permeability, these antibodies help reduce fluid leakage from blood vessels, which is beneficial in conditions like diabetic retinopathy and AMD, where excessive fluid leakage can cause vision problems. Clinically, anti-VEGF antibodies are used in cancer treatment to inhibit blood vessel growth that supplies tumors, effectively starving the tumor of nutrients and oxygen needed for growth. In ophthalmology, these therapies are injected directly into the eye to inhibit VEGF, thereby reducing pathological angiogenesis and fluid leakage in diseases such as AMD and retinal vein occlusion.

Examples of anti-VEGF antibodies include bevacizumab (Avastin), used primarily in oncology; ranibizumab (Lucentis), aflibercept (Eylea), and brolucizumab (Beovu) (used in ophthalmology. While effective, these therapies can have side effects such as local reactions (pain, redness, or infection at the injection site) and systemic effects (hypertension, thromboembolic events, and impaired wound healing). Over time, some tumors might develop resistance to anti-VEGF therapies, necessitating combination treatments or alternative strategies. While only bevacizumab is indicated for cancer treatment, it is anticipated that other anti-VEGF antibodies or fusion proteins can be effective in treating cancers.

### DETAILS OF THE INVENTION

RNAT92 comprises a variable light chain (Sequence 1), a variable heavy chain (Sequence No. 2), and transferrin protein conjugated through a peptide of ten to 25 amino acid chains as a non-cleavable linker. The single chain variable fragment is then linked to transferrin protein (P02787 TRFE_HUMAN) using either a cleavable or non-cleavable linker. (Table 1)

TABLE 1
The anti-VEGF VH and VL sequences of RNAT92

| No | Sequence |
|---|---|
| Sequence | EVQLVESGGGLVQPGGSLRLSCAASGYDFTHYGMNWVRQ |
| No: 1 | APGKGLEWVGWINTYTGEPTYAADFKRRFTFSLDTSKST |
| (VH) | AYLQMNSLRAEDTAVYYCAKYPYYYGTSHWYFDVWGQGT |
| | LVTVSS |
| Sequence | DIQLTQSPSSLSASVGDRVTITCSASQDISNYLNWYQQK |
| No: 2 | PGKAPKVLIYFTSSLHSGVPSRFSGSGSGTDFTLTISSL |
| (VL) | QPEDFATYYCQQYSTVPWTFGQGTKVEIK |

Designing and generating a single-chain fragment variable (scFv) antibody against VEGF-A requires choosing an appropriate linker, such as (G4S)3, as shown in Table 1; however, other linkers can also be used effectively.

Cleavable linkers are essential components in designing ocular drug delivery systems, facilitating the targeted release of therapeutic agents. Various cleavable linkers have been studied or used for ocular applications, each with unique mechanisms. Hydrazone linkers, for instance, cleave under acidic conditions, mimicking the lysosomal environment within ocular cells, which can help release small molecule inhibitors or peptides. Disulfide linkers, on the other hand, are cleaved by reducing agents such as glutathione, which are abundant in intracellular environments, making them suitable for delivering siRNA or DNA within ocular cells. A significant advantage of cleavable linkers is that once cleaved, the fusion protein with binding domains cannot return to circulation through the typical exocytosis process, leading to a longer half-life of the fusion protein in the eye.

Peptide linkers, cleaved by specific proteases found in the eye, can release anti-inflammatory or anti-angiogenic drugs. For example, linkers designed to respond to matrix metalloproteinases can be used in treating conditions like diabetic retinopathy. Enzyme-sensitive linkers, cleaved by enzymes upregulated in diseased ocular tissues, such as cathepsins or elastases, ensure targeted drug release in diseases like age-related macular degeneration. pH-sensitive linkers, which cleave under specific pH conditions, can release drugs in response to the slightly acidic environment of inflamed ocular tissues, which is helpful in conditions such as uveitis or glaucoma.

Photocleavable linkers, which cleave upon exposure to specific wavelengths of light, offer precise control over drug release timing and location, potentially valuable for surgical or diagnostic procedures. When designing cleavable linkers for ocular applications, considerations include ensuring biocompatibility to prevent toxicity to ocular tissues, stability to avoid premature drug release, and specificity to minimize off-target effects. These cleavable linkers enhance the efficacy and safety of treatments for various eye diseases, providing targeted and controlled drug delivery.

Noncleavable linkers are also crucial in designing ocular drug delivery systems, providing stable and sustained release of therapeutic agents without cleavage. These linkers maintain the integrity of the drug conjugate, ensuring that the therapeutic agent remains attached to the carrier throughout its journey to the target site.

Polyethylene glycol (PEG) linkers are widely used due to their biocompatibility and ability to prolong the circulation time of drugs. PEGylation can enhance the solubility and stability of drugs, making them ideal for treating chronic eye conditions where sustained release is beneficial. For example, PEGylated drugs can provide prolonged anti-inflammatory effects in diseases like uveitis or dry eye syndrome.

Amino acid linkers, composed of natural amino acids, offer another option for non-cleavable linkers. These linkers provide stability and can be tailored to achieve desired pharmacokinetic properties. In ocular drug delivery, amino acid linkers can stabilize peptides or proteins, ensuring that they remain intact and functional until they reach the target tissue. This is particularly useful for delivering therapeutic proteins in diseases like age-related macular degeneration or diabetic retinopathy. Amino acid linkers are widely utilized in bioconjugation due to their versatility, biocompatibility, and capacity for controlled release of therapeutic agents. Examples include the Glycine-Serine (Gly-Ser) linker, composed of repeating units of glycine and serine, which is flexible and hydrophilic, thus reducing steric hindrance and increasing solubility, making it ideal for fusion proteins and antibody-drug conjugates. The Glycine-Glycine-Phenylalanine-Glycine (GGFG) linker, which is cleavable by cathepsin B, an enzyme overexpressed in many tumors, ensures targeted drug release within the tumor microenvironment, commonly used in antibody-drug conjugates like Adcetris (brentuximab vedotin). Linkers containing acid-sensitive sequences such as aspartic acid facilitate intracellular release in acidic environments like endosomes or lysosomes, beneficial for drugs requiring activation within cells. Glutamic acid linkers are another option, cleavable by specific proteases or under certain conditions, suitable for targeted drug delivery systems. The Leucine-Serine (Leu-Ser) linker, with its balance of hydrophobic and hydrophilic properties, enhances the stability and solubility of conjugates, making it suitable for therapeutic proteins or antibodies. Additionally, the tripeptide linker Gly-Pro-Gly, known for its flexibility and resistance to proteolytic degradation, is often used in synthetic peptides and fusion proteins. These amino acid linkers provide a robust framework for bioconjugation, offering stability, specificity, and controlled release to enhance the efficacy and safety of therapeutic agents.

Polymeric linkers, made from synthetic polymers such as poly(lactic-co-glycolic acid) (PLGA), are also effective and non-cleavable. These polymers can form stable conjugates with drugs, providing a sustained release over an extended period. In ocular applications, PLGA linkers can be used in intravitreal injections to deliver anti-angiogenic agents, offering prolonged treatment for conditions like macular edema.

Noncleavable peptide linkers, which remain stable and do not degrade in the biological environment, can provide a continuous therapeutic effect. For instance, non-cleavable peptide linkers can attach therapeutic antibodies to carriers, ensuring that the antibodies remain functional and practical in neutralizing disease-causing agents in the eye.

When designing non-cleavable linkers for ocular applications, it is essential to consider biocompatibility, stability, and the ability to provide a sustained release. These linkers should not elicit an immune response or cause toxicity to ocular tissues. By providing a stable and continuous release of therapeutic agents, non-cleavable linkers enhance the efficacy and safety of treatments for various eye diseases, offering a reliable option for the long-term management of ocular conditions.

Suppose the linkers are made part of a recombinantly or mRNA-delivered product as a single molecule comprising the antibody and conjugated transferrin. In that case, a linker like the aspartic acid-proline (Asp-Pro) bond is suitable as it is susceptible to acidic environments due to the susceptibility of the peptide bond to acid-catalyzed hydrolysis. Histidine residues have a side chain with a pKa around 6.0, making them sensitive to pH changes near physiological and lysosomal conditions. However, incorporating multiple histidine residues in a row can create a segment in a peptide linker that becomes charged under acidic conditions, potentially destabilizing the linker and facilitating cleavage. Other examples of these linkers include:

Val-Cit (VC): The sequence Valine-Citrulline (Val-Cit) is a commonly used dipeptide linker that is cleavable by Cathepsin B. It is often used in antibody-drug conjugates (ADCs) targeting cancer cells.

Phe-Lys (FK): Phenylalanine-Lysine (Phe-Lys) is another dipeptide sequence that can be specifically cleaved by Cathepsin B. This linker is useful in contexts where a slightly different cleavage rate or stability is required compared to Val-Cit.

Gly-Phe-Leu-Gly (GFLG): This tetrapeptide sequence is one of the most typical Cathepsin B-sensitive linkers. It offers a balance of stability and efficient cleavage under physiological conditions.

Ala-Leu-Ala-Leu (Ala-Leu)2: This repeated dipeptide sequence provides a robust framework sensitive to Cathepsin B cleavage. It is helpful in formulations with extended linker lengths for optimal drug function.

The choice of non-cleavable peptide linkers will include G4S and other similar peptides that stay connected. Still, these would not be desirable except for connecting antibody fragments' VH and VL parts.

The present invention is an antibody-transferrin fusion protein that can readily cross the blood-retina barrier (BRB), allowing administration through systemic routes instead of the intravitreal route currently used for all such antibodies. The intravitreal route is painful, risky, and requires professional assistance, making it a high-cost and risky treatment; however, systemic administration has been impossible since the BRB prevents the entry of antibodies into the aqueous humor. Transferrin conjugation allows transcytosis using the iron transport receptors.

Transcytosis of antibodies and their fragments can occur through several mechanisms, including receptor-mediated transcytosis (RMT), adsorptive-mediated transcytosis, and cell-mediated transcytosis. One of the most exploited receptors for RMT is the transferrin receptor (TfR), which naturally facilitates iron transport into the eye. Antibodies designed to target the TfR can hitch a ride across the BRB through this mechanism.

Another target is the insulin receptor (IR), which, like the TfR, can mediate the transport of antibodies across the BRB, offering a pathway for therapeutic intervention. So, theoretically, a conjugate of insulin and an antibody should enhance the entry of antibodies across the BRB, though no such studies have been reported.

The low-density lipoprotein receptor-related protein-1 (LRP1) also serves as a conduit for delivering certain therapeutics into the eye, capitalizing on its role in transporting various molecules, including lipoproteins and amyloid-beta precursors.

Transferrin (Tf) is a serum protein that carries iron in the form of ferric ions (Fe3+). Tf has two binding sites for Fe3+; a non-bound Tf is called apo-Tf, while Tf bound to one Fe3+ is called mono-ferric, and two Fe3+ molecules di-ferric or holo-Tf (172). Tf-Fe3+ complexes are delivered to cells via transferrin receptor 1 (TfR1), expressed on the cell surface of most cells in the body. A second TfR has also been identified, TfR2, which has a lower affinity for Tf and is not as widely expressed, mainly in the liver. TfR1 is a transmembrane glycoprotein composed of two identical subunits of 90 kDa each, linked with disulfide bonds. Each subunit has three extracellular domains: the apical (A), the protease-like (P), and the helical domain (H). The helical domain has the main sites for holo-Tf. Holo-Tf binds with high affinity to TfR1 at physiological pH 7.4. After binding, the holo-Tf-TfR1 complex is internalized via clathrin-coated endosomes. The endosomes are acidified to pH 5.5, which results in dissociation from TfR1 and delivery of iron to the cell.

Antibodies can be engineered to bind to TfR directly or through transferrin. When engineering the antibody, it should preferably bind to a different epitope than Tf on the TfR to avoid interference with the endogenous process of iron delivery to the eye. Many TfR-antibodies bind the apical domain of TfR. It should have a moderate affinity for TfR in the nanomolar range. High affinity might result in a lower ability of the antibody to dissociate from TfR, which leads to sorting into lysosomes for degradation. The dissociation constant might be more critical than the association constant. Too low affinity might lead to poor ability of the antibody to bind TfR at the BRB and, consequently, poor retinal delivery. Affinity can be pH-dependent so that the antibody binds TfR well at physiological pH but can dissociate at lowered pH in the early endosome.

The dose of the antibody is also an essential factor. Higher affinity can increase retinal uptake at low doses, while for high doses, as used in therapy, a lower affinity for TfR increases eye uptake. At non-saturable low doses, binding and eye uptake depends on affinity. In contrast, at saturable doses, lower affinity could be critical for the dissociation from TfR and entry to the eye.

The valency of the antibody to TfR is crucial. A monovalent interaction with TfR is preferable for high parenchymal delivery. A bivalent binding to TfR is more robust due to high avidity. It could cause clustering of TfR on the capillary cells and lead to intracellular sorting to the lysosomes rather than transcytosis. A bivalent antibody can also decrease TfR levels in cells.

Conjugating with transferrin offers the best option of all the choices available for RMT. There are several considerations in designing this conjugate. First, the suitable conjugation site should be identified on both the antibody and transferrin to enable access to form a stable linkage without compromising the function of either protein. Commonly targeted sites on antibodies include lysine residues that are abundant on the surface and reactive due to their amine groups. Thiol groups are less plentiful but can be used. Two ways an antibody or a fragment can be conjugated are by allowing the engineered antibody to bind with transferrin in vivo and chemically binding the antibody and transferrin protein.

Iron, insoluble as free Fe3+ and toxic as free Fe2+, is distributed through the body as Fe3+ bound to transferrin (Tf) for delivery to cells by endocytosis of its complex with transferrin receptor (TfR). Transferrin has two specific iron-binding sites in the N-lobe (N-terminal lobe) and C-lobe (C-terminal lobe). Each site can reversibly bind one ferric ion (Fe\hat{ }3+) along with a carbonate anion and function independently. The N-Lobe (Transferrin A) is part of the N-terminal lobe of transferrin. It binds iron when transferrin interacts with its receptor on the surface of cells, specifically in areas where the pH is slightly more acidic, facilitating iron release. The C-Lobe (Transferrin B) is located in the C-terminal lobe; this site also binds iron under similar conditions but functions independently, meaning the iron release and binding can occur at either site without necessarily affecting the other.

Transferrin binding of antibodies can expedite entry into cancer cells by exploiting the transferrin receptor (TfR), which is frequently overexpressed on the surface of many cancer cells. The transferrin receptor mediates iron uptake by binding transferrin, an iron-carrying protein. Still, researchers have adapted this pathway to enhance the delivery of therapeutic agents, including antibodies, into cancer cells. This method offers several advantages, including enhanced uptake, selective targeting, and improved intracellular delivery. With their high metabolic activity and rapid proliferation, cancer cells often exhibit an increased demand for iron, resulting in the overexpression of transferrin receptors and providing more binding sites for transferrin-conjugated antibodies. This increased binding facilitates the more efficient uptake of therapeutic agents. Furthermore, since transferrin receptors are more abundantly expressed in cancer cells than normal cells, this strategy allows for more selective targeting of cancer cells, potentially reducing off-target effects and improving the therapeutic index of the antibody. Once the transferrin-conjugated antibody binds to the transferrin receptor on the cancer cell surface, the receptor-antibody complex is internalized into the cell via endocytosis, enhancing the intracellular delivery of the antibody and enabling it to reach its intracellular targets more effectively.

As a result, antibodies like ranibizumab, brolicuzimab, and fusion protein aflibercept can be used to treat cancer, even though they are not approved for this indication.

The RNAT92 can be manufactured using standard recombinant technology or delivered in vivo through mRNA. The RNAT92 can be administered intravenously, subcutaneously, or intramuscularly. The number of protein molecules generated from a single mRNA is primarily determined by “translation efficiency.” The stability of the mRNA molecule, the availability of different translation components, and the existence of translation initiation sites are some factors affecting translation efficiency. The heterologous peptides are often used as signal sequences to direct the synthesized protein to specific locations within the cell, such as the endoplasmic reticulum (ER) or extracellular space.

In preferred embodiments, the RNAT92 is an antibody that binds to VEGF upon entering the vitreal fluid upon systemic administration.

A second aspect relates to the manufacturing of RNAT92 by either standard recombinant expression or mRNA encoding.

The fusion protein is administered, preferably intravenous, subcutaneous, intramuscular, or intradermal administration of RNAT92. This results in blood concentration that yields sufficient transfer across the BRB to create a clinically effective response.

Preferably, the mRNA encoding involves the delivery of mRNA in a lipid nanoparticle that also acts as an adjuvant.

1. An anti-VEGF single chain fusion protein (RNAT92) comprising at least one variable heavy chain and at least one variable light chain, both linked together with a non-cleavable linker and further connected to a transcytosis agent with a cleavable or non-cleavable linker.

2. The anti-VEGF single chain fusion protein (RNAT92) of claim 1, wherein the variable heavy chain is Sequence No. 1.

3. The anti-VEGF single chain fusion protein (RNAT92) of claim 1, wherein the variable light chain is Sequence No. 2.

4. The anti-VEGF single chain fusion protein (RNAT92) of claim 1, wherein the transcytosis agent is transferrin protein (P02787 TRFE_HUMAN).

5. The anti-VEGF single chain fusion protein (RNAT92) of claim 1, wherein the non-cleavable linker is an amino acid, polyethylene glycol, or a polymer, preferably glycine-glycine-glycine-glycine-serine (G4S), or its multiples thereof.

6. The anti-VEGF single chain fusion protein (RNAT92) of claim 1, wherein the cleavable linker is hydrazone, disulfide, or peptide, preferably Gly-Gly-Phe-Gly (GGFG), or its multiples thereof.

7. The anti-VEGF single chain fusion protein (RNAT92) of claim 1, wherein the RNAT92 comprises Sequence No. 1-(G4S)3-Sequence No. 2-(G4S)3-P02787 TRFE_HUMAN.

8. The anti-VEGF single chain fusion protein (RNAT92) of claim 1, wherein the RNAT92 comprises Sequence No. 1-(G4S)3-Sequence No. 2-GGFG-P02787 TRFE_HUMAN.

9. The anti-VEGF single chain fusion protein (RNAT92) of claim 1, wherein the RNAT is capable of inhibiting the activation of VEGF receptors, decreasing neovascularization in the eye, and thus treating Neovascular Age-related Macular Degeneration (nAMD or wet AMD or wAMD) and for Diabetic Macular Edema (DME).

10. The anti-VEGF single chain fusion protein (RNAT92) of claim 1, wherein RNAT92 is capable of treating cancer or enhancing the activity of other anticancer drugs.

11. The anti-VEGF single chain fusion protein (RNAT92) of claim 1, wherein RNAT92 is administered by intravitreal, intramuscular, subcutaneous, or intravenous route.

12. The anti-VEGF single chain fusion protein (RNAT92), wherein the RNAT92 is manufactured using recombinant protein technology in bacteria or a mammalian cell.

13. The anti-VEGF single chain fusion protein (RNAT92), wherein the RNAT92 is delivered using an mRNA capable of encoding RNAT92 in vivo in a lipid nanoparticle formulation.


SEQUENCE LISTING (2 sequences)

SEQ ID NO: 1 - protein, 123 aa, Homo sapiens [sequence omitted]

SEQ ID NO: 2 - protein, 107 aa, Homo sapiens [sequence omitted]